(19) United States
(12) Patent Application Publication
(10) Pub. No.: US 2005/0129575 A1
(43) Pub. Date: Jun. 16, 2005

(54) Heavy metals monitoring apparatus

(75) Inventors: James A. Partridge, Waynesville, NC (US); James E. Partridge, Waynesville, NC (US); Jeffery S. Partridge, Venice, FL (US); D. Ingram Gillam, Asheville, NC (US)

Correspondence Address: STEVEN C. SCHNEDLER;CARTER SCHNEDLER & MONTEITH, PA, 56 CENTRAL AVE., SUITE 101, PO BOX 2985, ASHEVILLE, NC, 28802, US

(73) Assignee: CHEMTRONIX INC., Waynesville, NC (US)

(21) Appl. No.: 10/987,579

(22) Filed: Nov. 12, 2004

Related U.S. Application Data
(60) Provisional application No. 60/519,598, filed on Nov. 12, 2003.

(51) Int. Cl. G01N 33/00

(52) U.S. Cl. 422/62; 436/55

(57) ABSTRACT

Apparatus and methods for monitoring of dissolved (ionic) heavy metal residuals in liquid process stream or liquid waste (effluent) streams, as examples, after recovery and collection of such metals from a laboratory, medical, industrial or manufacturing process stream. An automatic controller is employed to deliver a sample volume of liquid from the stream to a measurement chamber, adjust the conductivity of the sample volume within the measurement chamber to a predetermined conductivity at a predetermined conductivity measurement temperature, employ an ion specific electrode and a reference electrode to make a voltage measurement representative of the concentration of specific metal ions within the sample volume at the predetermined concentration measurement temperature, record the voltage measurement, and flush the measurement chamber.

### CROSS-REFERENCE TO RELATED APPLICATION

The benefit of U.S. provisional patent application Ser. No. 60/519,598, filed Nov. 12, 2003, is claimed. The entire disclosure of patent application Ser. No. 60/519,598 is hereby expressly incorporated by reference.

### BACKGROUND OF THE INVENTION

The invention relates generally to the monitoring of dissolved (ionic) heavy metal residuals in liquid process stream or liquid waste (effluent) streams, as examples, after recovery and collection of such metals from a laboratory, medical, industrial or manufacturing process stream.

In the United States, the Federal government, through Environmental Protection Agency (EPA) regulations, has issued stringent national compliance standards for the discharge of many heavy metals into sanitary sewers. For example, one such EPA regulation stipulates that no more than 5 ppm silver may be discharged into a sanitary sewer. However, operation of medical X-ray film processing devices, in particular those incorporating photographic fixers, typically generate large amounts of ionic silver and ionic silver complexes as by-products to the photographic process. This is also true for most bleach fix color photo systems, such as those found at photo studios and in photo departments located within retail stores and many of the larger drug stores.

Silver recovery units normally are attached to X-ray film and color photo-processors to capture silver from their spent fixer. Over time, such silver recovery units degrade in function and, as a result, it is common for liquids containing 50-1000 ppm silver to be discharged to waste drains connected to municipal sanitary sewers. Even the lower number (50 ppm) within this range well exceeds the EPA-mandated 5 ppm silver limit.

For the most part, this mandate has been largely ignored by the medical and photographic communities because heretofore there has been no practical way to monitor the effluent silver in real time or near real time. Without the ability to trace discharges, elimination of ongoing excessive silver through timely and appropriate process equipment changes becomes difficult or impossible. The result has been (and continues to be) silver finding its way to the local Publicly Owned Treatment Works (POTW) (or sewer plant), where it can potentially cause severe problems as a result of the destruction of the many microbiologicals necessary for the proper digestion of human waste material.

There are similar problems with monitoring the recovery process associated with other heavy metal waste streams, such as those found in the manufacture of printed circuit boards (copper and silver), batteries (lead), and the electroplating industry (copper, silver, zinc, tin and nickel).

### SUMMARY OF THE INVENTION

In one aspect, a method is provided for monitoring concentration of ions of a specific metal in solution in a liquid process or liquid waste stream. An automatic controller is employed to deliver a sample volume of liquid from the stream to a measurement chamber, adjust the conductivity of the sample volume within the measurement chamber to a predetermined conductivity at a predetermined conductivity measurement temperature, employ an ion specific electrode and a reference electrode to make a voltage measurement representative of the concentration of specific metal ions within the sample volume at the predetermined concentration measurement temperature, record the voltage measurement, and flush the measurement chamber.

In another aspect, a method is provided for re-standardizing an ion specific electrode. For predetermined durations, voltages first of one polarity and then of opposite polarity are applied to the ion specific electrode with reference to a re-standardizing reference electrode.

In yet another aspect, apparatus for monitoring concentration of ions of a metal in solution in a liquid process or liquid waste stream is provided. The apparatus includes a measurement chamber, a conductivity sensor within the measurement chamber, an ion specific electrode and a reference electrode within the measurement chamber, a controlled sample injection pump for delivering liquid from the stream to the measurement chamber. a controlled ISA injection pump for delivering an ionic strength adjuster liquid to the measurement chamber, a controlled water pump for delivering water to the measurement chamber, and a controller electrically connected to receive signals from the conductivity sensor and from the ion specific and reference electrodes and connected to control operation of the sample injection pump, the ISA injection pump and the water pump. The controller is operable to effect a measurement cycle by directing the sample injection pump to deliver a sample volume of liquid from the stream to the measurement chamber, adjusting the conductivity of the sample volume within the measurement chamber to a predetermined conductivity at a predetermined conductivity measurement temperature by employing the conductivity sensor and the ISA injection pump in a feedback loop, making a voltage measurement representative of the concentration of specific metal ions in the sample volume at a predetermined concentration measurement temperature by employing the ion specific electrode and the reference electrode, and recording the voltage measurement, and flushing the measurement chamber.

In still another aspect, a system for monitoring concentration of ions of a metal in solution in a liquid process or liquid waste stream is provided. The system includes a local or client device, and a remote or server computer device. The local device includes a measurement chamber, a conductivity sensor within the measurement chamber, an ion specific electrode and a reference electrode within the measurement chamber, a controlled sample injection pump for delivering liquid from the stream to the measurement chamber, a controlled ISA injection pump for delivering an ionic strength adjuster liquid to the measurement chamber, a controlled water pump for delivering water to the measurement chamber, and a controller electrically connected to receive signals from the conductivity sensor and from the ion specific and reference electrodes and connected to control operation of the sample injection pump, the ISA injection pump and the water pump. The controller is operable to effect a measurement cycle by directing the sample injection pump to deliver a sample volume of liquid from the stream to the measurement chamber, adjusting the conductivity of the sample volume within the measurement chamber to a predetermined conductivity at a predetermined conductivity measurement temperature by employing the conductivity sensor and the ISA injection pump in a feedback loop, making a voltage measurement representative of the concentration of specific metal ions in the sample volume at a predetermined concentration measurement temperature by employing the ion specific electrode and the reference electrode, and recording the voltage measurement, and flushing the measurement chamber. The local or client device also includes a local communications device for transmitting the voltage measurement to a remote location for recording. The remote computer device at the remote location receives and records the voltage measurement.

### BRIEF DESCRIPTION OF THE DRAWINGS

FIG. 1 is an overall representation of apparatus embodying the invention;

FIG. 2 is a view of the front panel of an enclosure for the electronic subsystem of FIG. 1;

FIG. 3 is a functional diagram particularly of components within the fluid-handling subsystem of FIG. 1;

FIG. 4 is an enlarged exploded view of the measurement chamber which is part of the fluid-handling subsystem of FIG. 3;

FIG. 5 is a representational block diagram particularly of the electronic subsystem of FIG. 1, but also including components within the fluid-handling subsystem;

FIG. 6 is a diagram, partially in electrical schematic form and partially in block diagram form, of the temperature sensing and conductivity subsystems shown in FIG. 5;

FIG. 7 is a diagram, partially in electrical schematic form and partially in block diagram form, of the concentration sensing and heating subsystems of FIG. 5;

FIG. 8 is a diagram, partially in electrical schematic form and partially in block diagram form, of various additional control and output subsystems represented in FIG. 5;

FIG. 9 is a program flowchart representing Initialization and Main Loop software routines programmed within the microcontroller of FIGS. 5-8;

FIG. 10 is a program flowchart representing a “CALLHOME” software routine programmed within the microcontroller of FIGS. 5-9;

FIG. 11 is a program flowchart representing a “SILVERCYCLE” software routine programmed within the microcontroller of FIGS. 5-8;

FIG. 12 is a program flowchart representing a “NEWSTAT” software routine programmed within the microcontroller of FIGS. 5-8;

FIG. 13 is a program flowchart representing a “HEATUP” software routine programmed within the microcontroller of FIGS. 5-8;

FIG. 14 is a program flowchart representing a “HOLDING” software routine programmed within the microcontroller of FIGS. 5-8;

FIG. 15 is a program flowchart representing a “CLEARALL” software routine programmed within the microcontroller of FIGS. 5-8;

FIG. 16 is a program flowchart representing a “QWIKSTAT” software routine programmed within the microcontroller of FIGS. 5-8;

FIG. 17 is a program flowchart representing a “CLEANPROBE” software routine programmed within the microcontroller of FIGS. 5-8;

FIG. 18 is a program flowchart representing a “TMPCAPCHRGE” software routine programmed within the microcontroller of FIGS. 5-8;

FIG. 19 is a program flowchart representing a “CONDUCTREAD” software routine programmed within the microcontroller of FIGS. 5-8;

FIG. 20 is a program flowchart representing a “GETTEMPERATURE” software routine programmed within the microcontroller of FIGS. 5-8;

FIG. 21 is a program flowchart representing a “DWELLDELAY” software routine programmed within the microcontroller of FIGS. 5-8;

FIG. 22 is a program flowchart representing a “RINSEFILLCYCLE” software routine programmed within the microcontroller of FIGS. 5-8; and

FIG. 23 is a program flowchart representing a “DUMPCYCLE” software routine programmed within the microcontroller of FIGS. 5-8.

### DETAILED DESCRIPTION

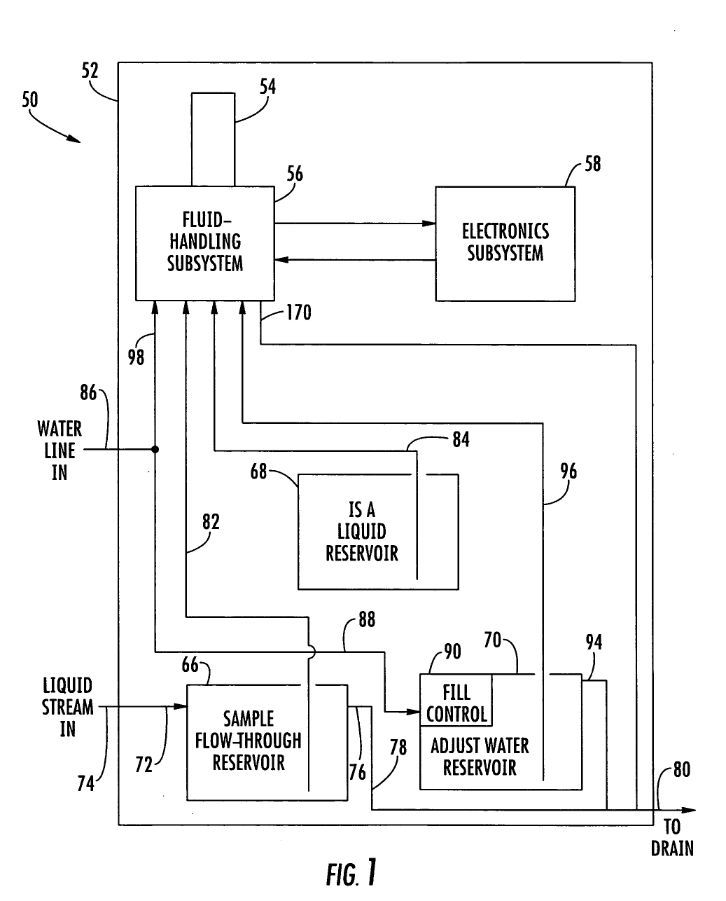

Referring first to FIG. 1, apparatus embodying the invention for monitoring concentration of ions of a metal in solution in a liquid process or liquid waste stream is generally designated 50. In the embodiment of FIG. 1, the apparatus 50 takes the form of a self-contained unit 52, which is representative of a wheeled cart including shelves (not shown) for convenient and accessible support of various liquid reservoirs described hereinbelow, as well as for mounting of various other subsystems and components described hereinbelow. Moreover, as the self-contained unit 52, the apparatus 50 is suitable for location within a photo studio or within a photo department located within a retail store, such as a drugstore.

In general, apparatus and methods embodying the invention are suitable for analyzing liquid process streams or liquid waste (effluent) streams in a short-term batch process mode so that near real-time data is available to monitor the concentration of heavy metal ions within liquid being discharged to a drain. As described in detail hereinbelow, a microprocessor-based controller stores this data locally and, in a client/server system, sends data by means of an internal modem to a computer site (or sites) within the test facility, as well as to outside locations of choice. Diagnostic information can also be forwarded to a monitoring unit provider (via the same internal modem) to track performance parameters, facilitating the scheduling of timely and appropriate maintenance and/or service work as required. Although the specific embodiment disclosed herein is for monitoring the concentration of silver ions in spent fixer in X-ray film and color photo-processors, the apparatus and methods of the invention are also applicable to measuring the ionic concentration of other heavy metals such as copper, nickel and lead, which are present in other and completely unrelated industries.

The apparatus 50 is intended to be employed in combination with a separate processing device, such as a metal recovery device, not specifically shown. As a more particular example, the apparatus 50 may be employed in combination with a silver recovery system attached to a photo-processing unit in order to monitor the discharge from the silver-recovery unit. Moreover, as is described hereinbelow, the apparatus 50 optionally may be configured to positively shut down the operation of the associated equipment in the event heavy metal ion concentration becomes excessive. The apparatus 50 accordingly facilitates the monitoring of the discharge of various heavy metals, such as silver discharged from medical X-ray and color film and photo devices, in a manner that allows the metal recovery apparatus/process to comply with current EPA regulations.

The apparatus 50 more particularly includes a hollow cylindrical measurement chamber 54, a fluid-handling subsystem 56 of which the measurement chamber 54 actually forms a part, and an electronics subsystem 58. A lower portion of the measurement chamber 54 lies slightly within the housing of the fluid-handling subsystem 56, simplifying various connections, described hereinbelow, between the fluid-handling subsystem 56 and the measurement chamber 54.

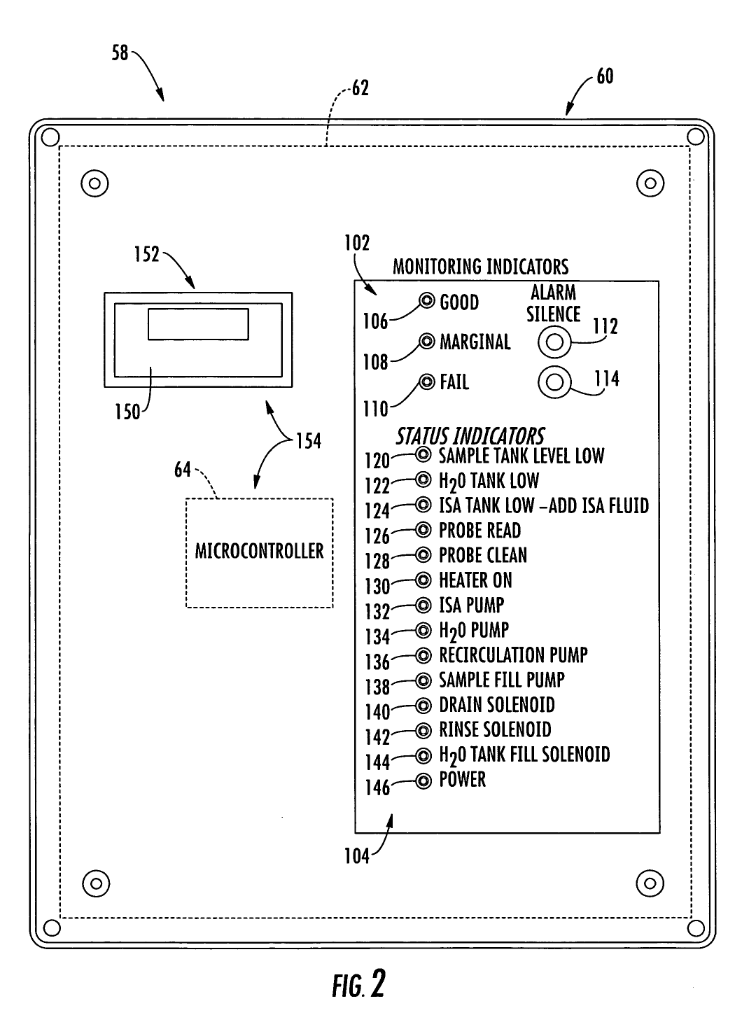

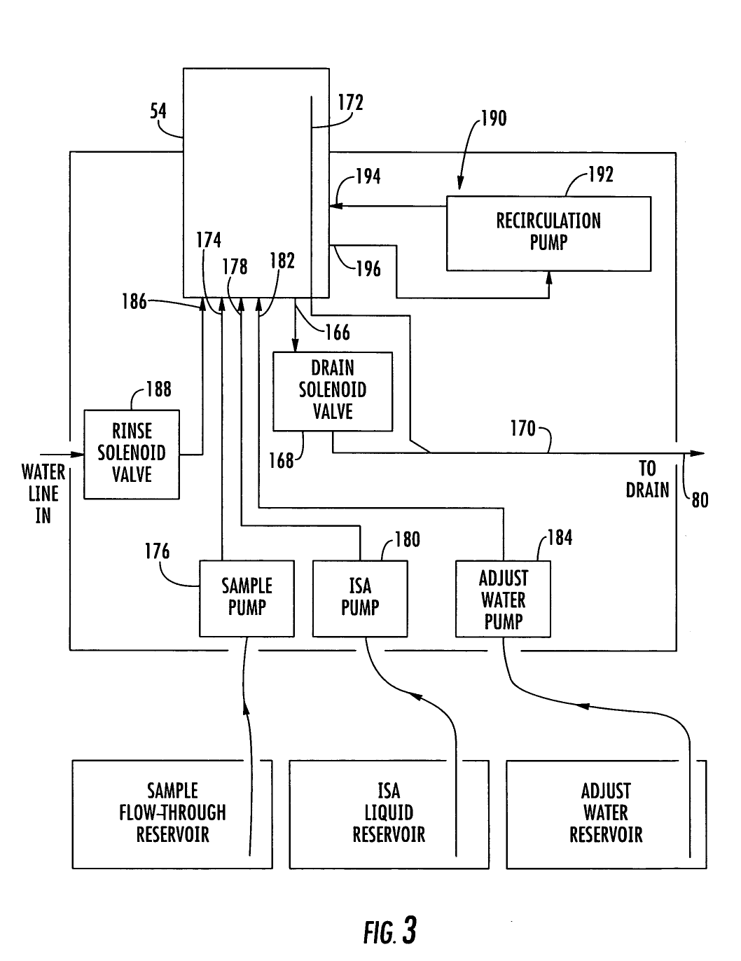

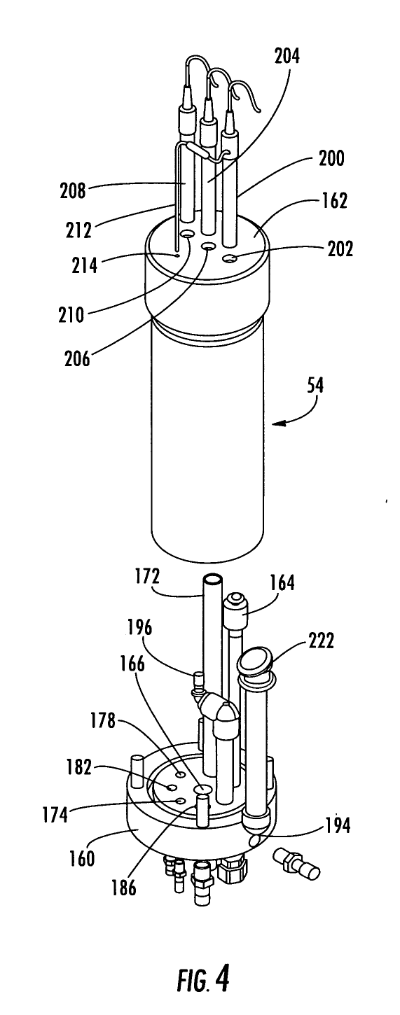

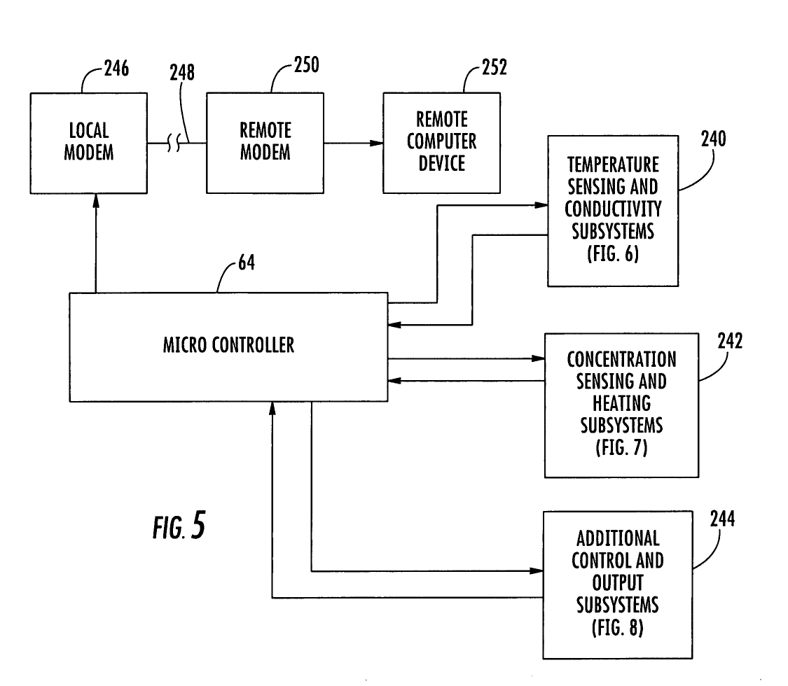

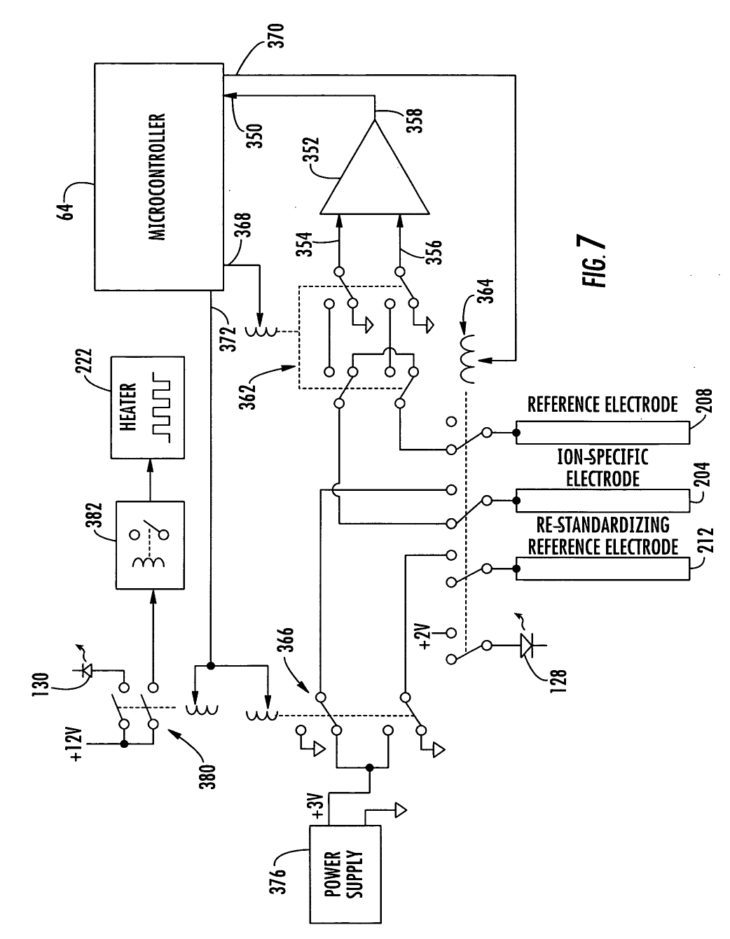

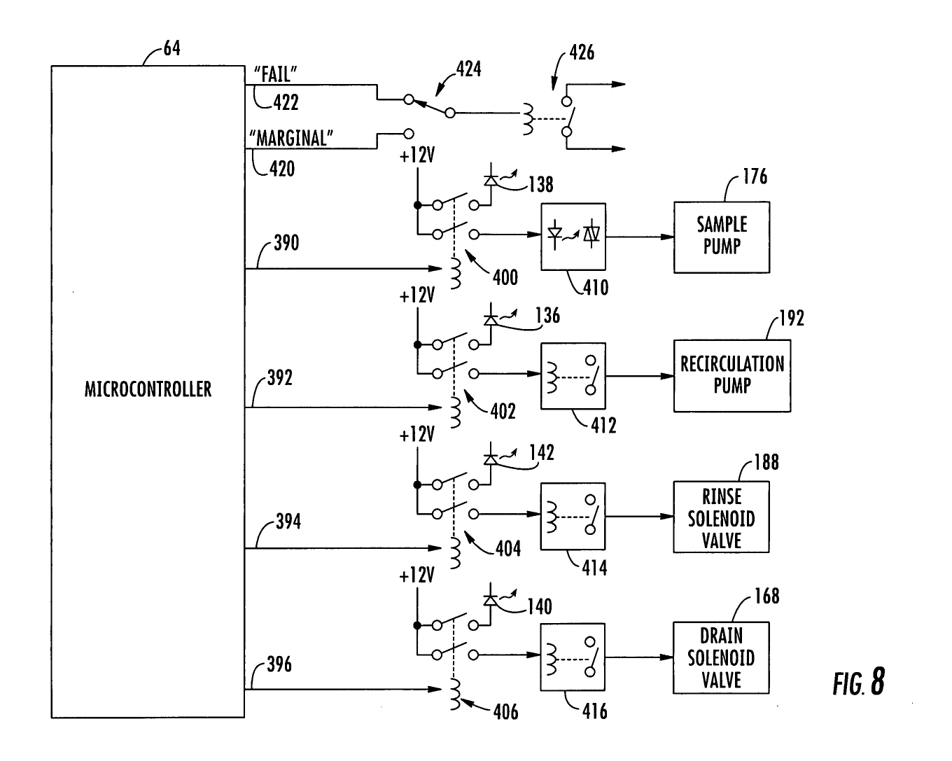

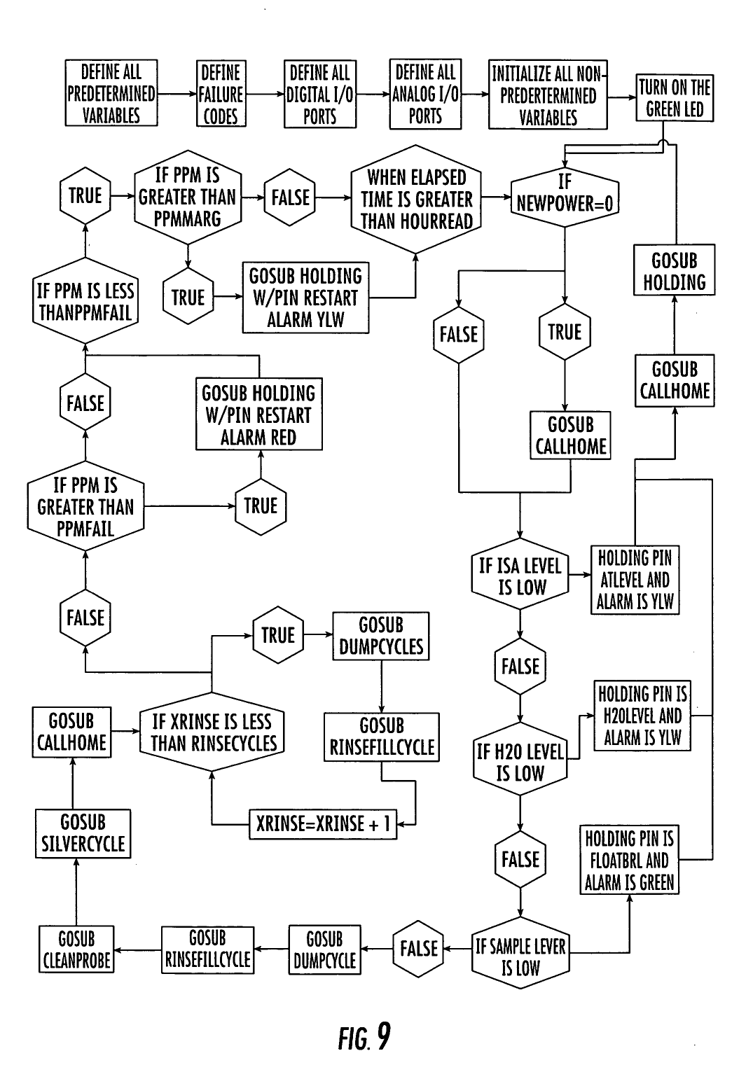

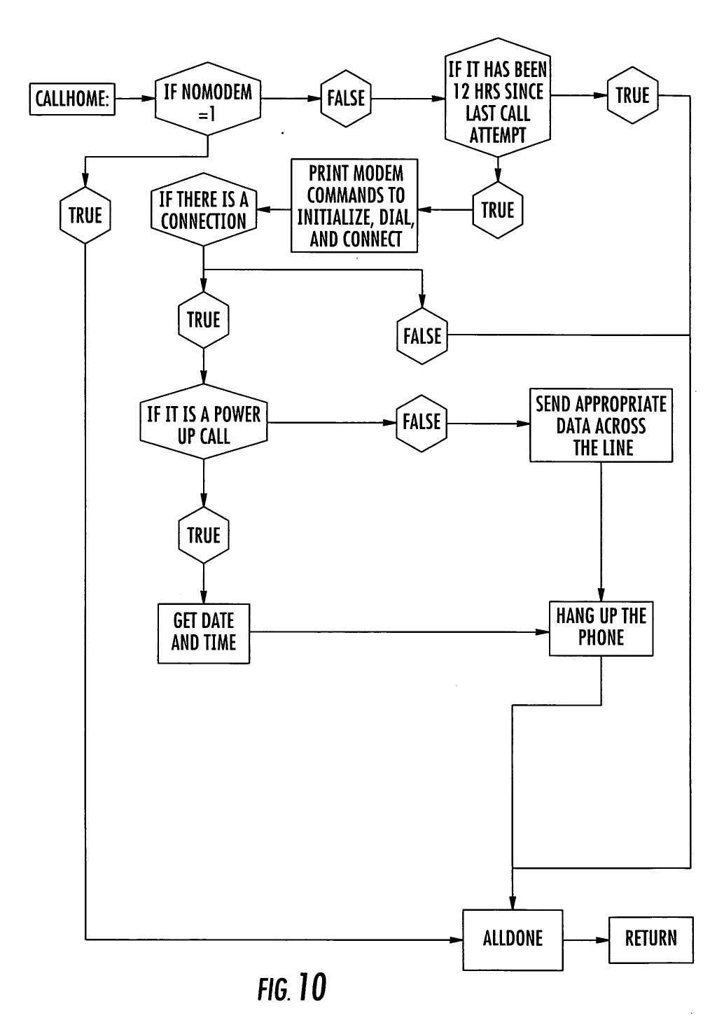

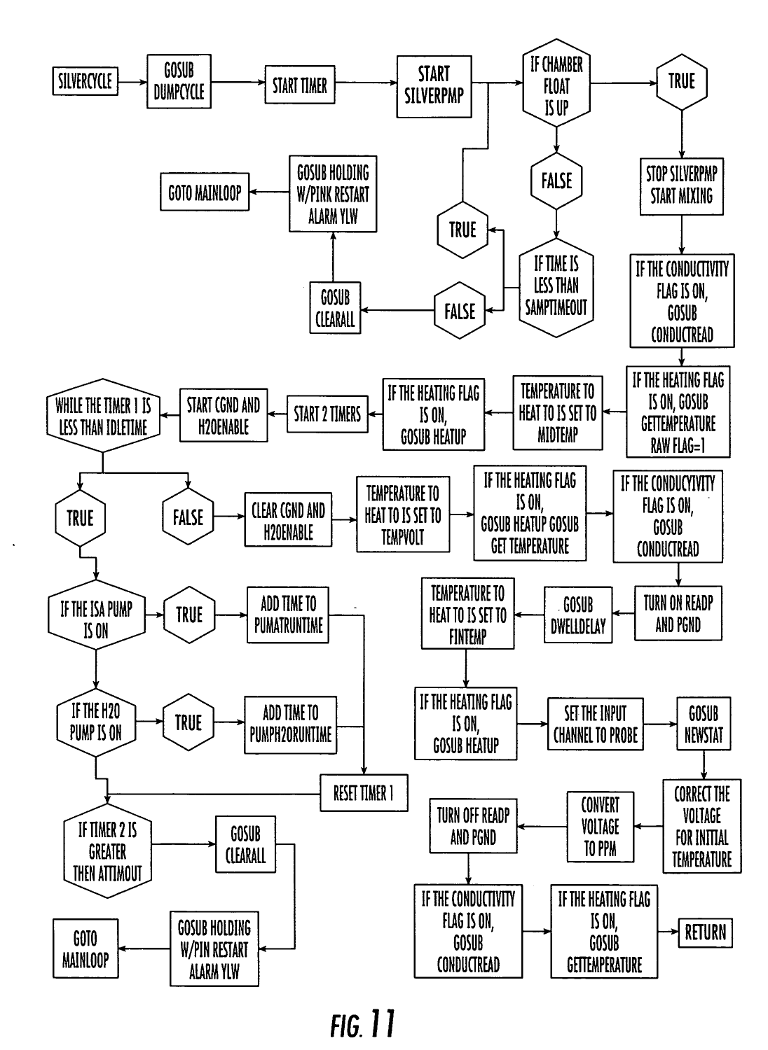

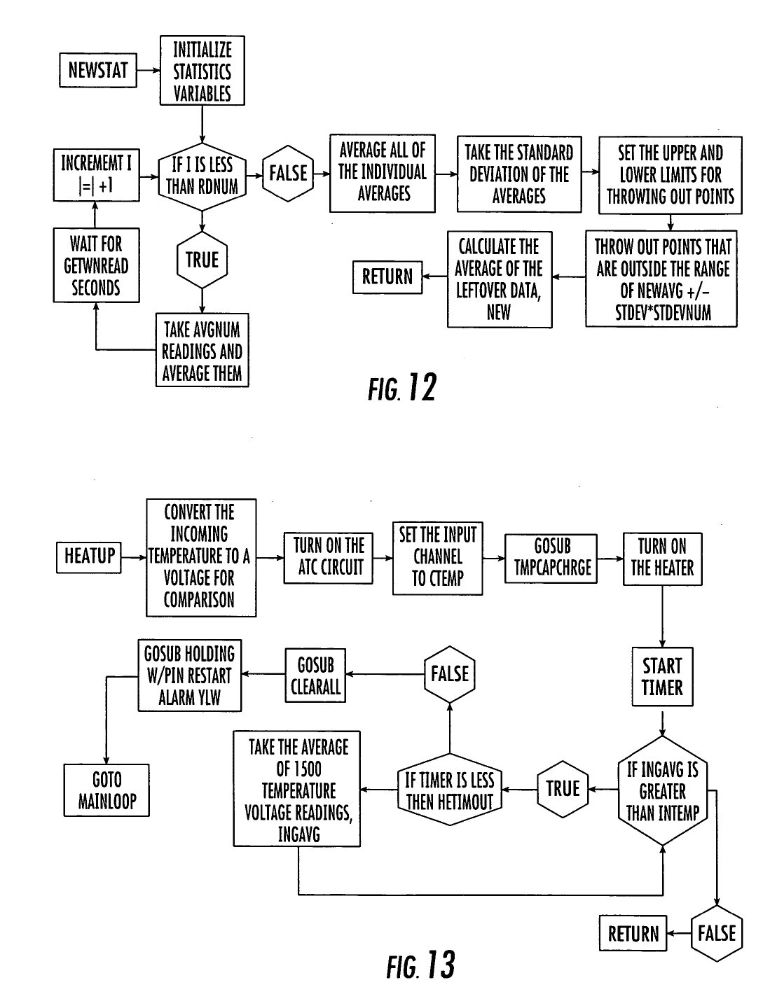

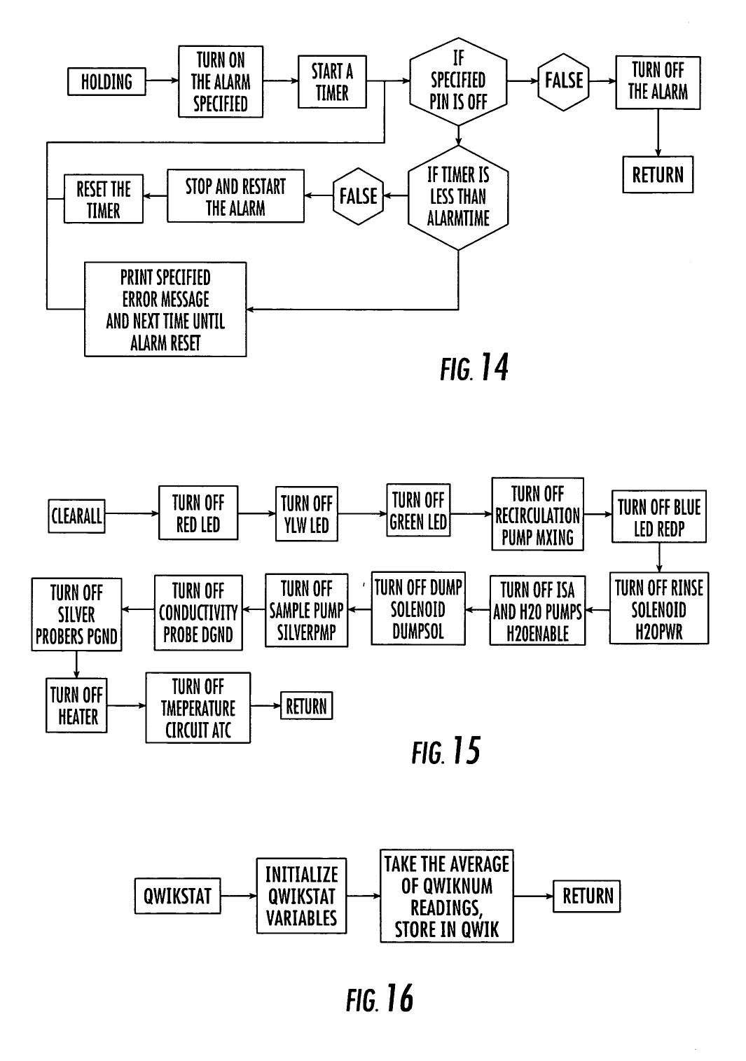

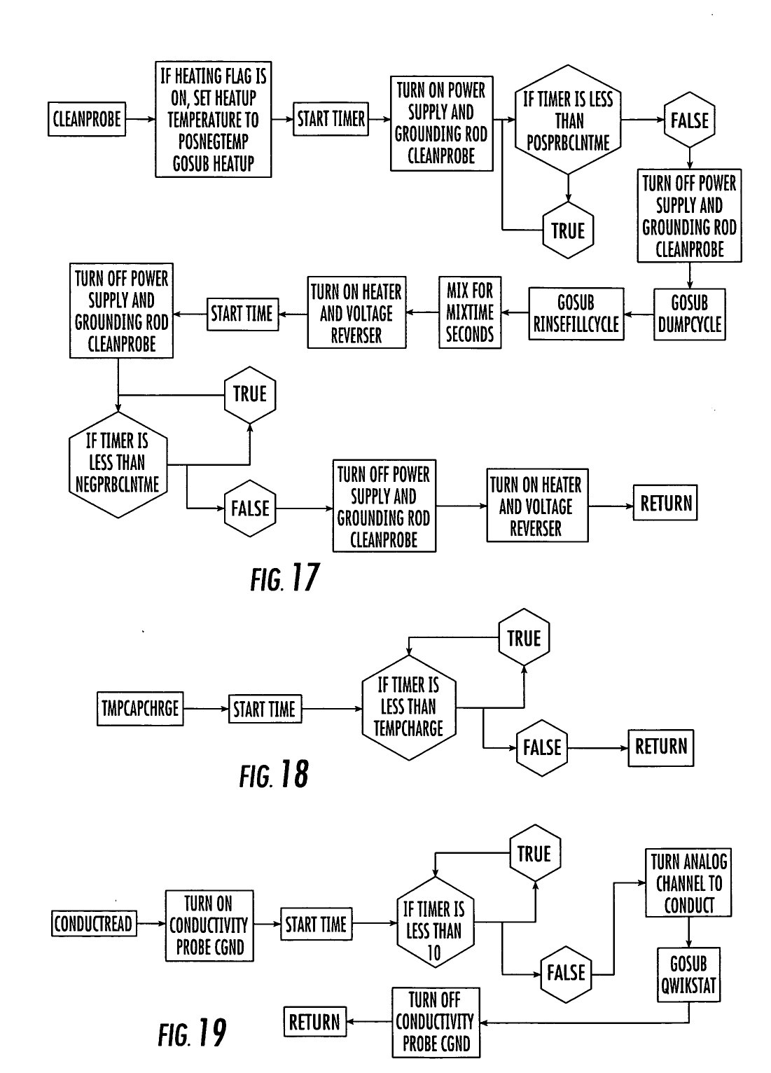

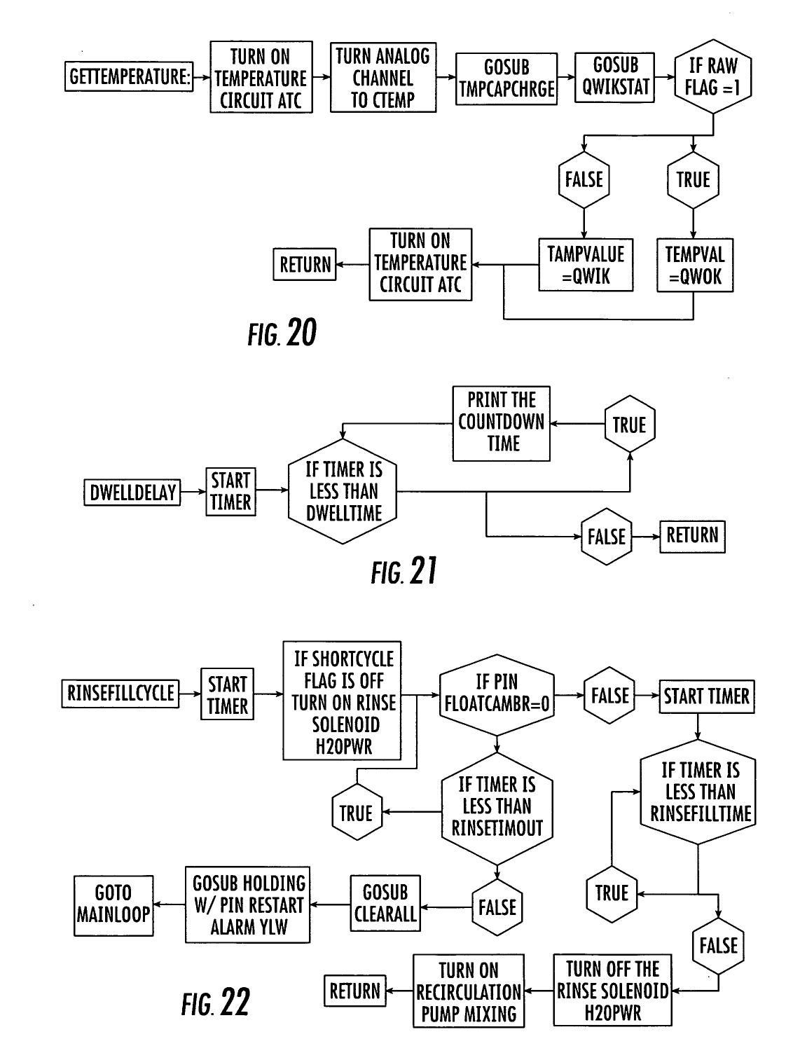

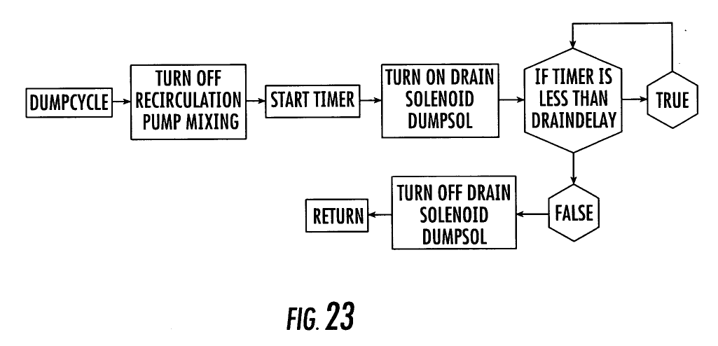

The fluid-handling subsystem 56 is generally contained within a plastic hinged-cover case (not shown), and contains various electromechanical components, in particular, pumps and solenoid valves, described hereinbelow with reference to FIGS. 3 and 4, as well as interconnecting tubing. The electronics subsystem 58 likewise is generally contained within a hinged-cover case 60 (FIG. 2), which houses electronic printed circuit boards such as a mother board 62 represented in dash lines in FIG. 2, and various components mounted thereon. Included on the mother board 62 is a microcontroller 64 which, in general, directs the overall operation of the apparatus 50 under program control, including in particular the various electromechanical components of the fluid-handling subsystem 56. The microcontroller 64 and its operation are described in greater detail hereinbelow with reference to FIGS. 5-8, and computer program code stored therein is represented in flowchart form in FIGS. 9-23.

With reference still to FIG. 1, three reservoirs are provided, each of corrosion-resistant plastic construction. In particular, there are a sample flow-through reservoir 66, an ISA liquid reservoir 68, and an adjust water reservoir 70. In the embodiment of FIG. 1, the reservoirs 66, 68 and 70 each have a liquid capacity of five gallons.

The sample flow-through reservoir 66 has an input 72 for receiving a liquid process stream or liquid waste stream represented at 74, as well as an overflow outlet 76 which discharges via an outlet line 78 to a drain line 80.

Thus, during operation, the sample flow-through reservoir 66 remains nearly full of liquid representative of current discharge of the associated equipment, such as photo-processing equipment. Connected so as to enable liquid to be drawn from a lower portion of the sample flow-through reservoir 66 is a sample line 82, the other end of which is connected to the fluid-handling subsystem 56.

The ISA liquid reservoir 68 contains, and is periodically manually refilled with, an ionic strength adjuster (ISA) liquid appropriate to the chemistry of the liquid stream being monitored, and to the specific metal whose ionic concentration is being monitored. In an apparatus 50 for monitoring the concentration of silver ions in spent photographic fixer, an ammonium thiosulfate solution may be employed as the ionic strength adjuster. An ISA line 84 is positioned so as to draw ISA liquid from near the bottom of the ISA liquid reservoir 68, the other end of which is connected to the fluid-handling subsystem 56.

The adjust water reservoir 70 contains ordinary tap water piped in via a water line represented at 86, and has as its primary purpose the warming of potentially cold tap water to a temperature approaching room temperature for adjusting sample electrical conductivity as described hereinbelow, rather than directly using cold incoming tap water for that purpose.

The incoming tap water line 90 is connected via a connecting line 88 to a fill control subsystem, generally designated 92. In the embodiment described herein, the fill control subsystem comprises a pair of level sensors (not shown) within the adjust water reservoir 70 and connected to simple circuitry (not shown) within the electronics subsystem 58 for activating a solenoid valve (not shown) to allow tap water to flow into the adjust water reservoir 70 when the water level falls to the height to the lower sensor, and to deactivate the water fill solenoid (not shown) when the water level within the adjust water reservoir 70 reaches the upper sensor. A mechanical float valve, such as is found within a toilet tank, may as well be employed. As a precaution in the event of malfunction of the fill control subsystem 92, the adjust water reservoir has a overflow outlet 94, connected to the drain line 80.

For drawing adjust water as needed from the adjust water reservoir 70, an adjust water line 96 is connected for drawing water from the near the lower end of the adjust water reservoir 70. The other end of the adjust water line 96 is connected to the fluid-handling subsystem 56.

Each of the reservoirs 54, 68 and 70 includes a “nearly empty” sensor (not shown) connected to circuitry within the electronic subsystem 58 in order to signal that the apparatus 50 should be shut down in the event a liquid necessary for operation is not present.

The water line 86 is additionally connected via another line 98 directly to the fluid-handling subsystem 56 to provide rinse water for the measurement chamber 54, as described hereinbelow. Cold incoming tap water is acceptable for purposes of rinsing the measurement chamber 54, and conserves the quantity of water warmed to room temperature within the adjust water reservoir 70.

Referring again to FIG. 2, the front panel of the electronics subsystem 58 includes an indicator subpanel 100 including a set of monitoring indicators, generally designated 102, and a set of status indicators, generally designated 104. The various indicators may take the form of light emitting diodes (LEDs). The monitoring and status indicators 102 and 104 generally reflect operation of the apparatus 50, and permit an observer to quickly verify proper operation (or not). More particularly, the monitoring and status indicators 102 and 104 show real-time Pass/Fail monitoring information as well as the activity status of all major monitoring functions.

The set of monitoring indicators 102 more particularly includes a “good” LED 106, a “marginal” LED 108 and a “fail” LED 110. Associated with the “marginal” and “fail” LEDs 108 and 110 are alarm silence pushbuttons 112 and 114, respectively.

The set of status indicators 104 more particularly includes a “sample tank low” LED 120; an “H₂O tank low” LED 122; an “ISA tank low” LED 124, a “probe read” LED 126; a “heater on” LED 130; an “ISA pump” LED 132; an “H₂O pump” LED 134; a “recirculation pump” LED 136; a “sample fill pump” LED 138; a “drain solenoid” LED 140; a “rinse solenoid” LED 142; an “H₂O tank fill solenoid” LED 144; and a “power” LED 146. Connections to various ones of these LEDs are more shown in the partial electrical schematic diagrams of FIGS. 6-8 described hereinbelow.

Also visible in FIG. 2 is the front panel 150 of a conductivity controller 152, described in greater detail hereinbelow with reference to FIG. 6. The microcontroller 64 and the conductivity controller together comprise an automatic controller.

With reference now to FIGS. 3 and 4, in the illustrated embodiment the measurement chamber 54 more particularly takes the form of a hollow cylindrical column, oriented vertically, made of PVC plastic, as an example. The plastic material may be either clear or opaque. The cylindrical measurement chamber 54 is approximately four inches in diameter and fourteen inches tall. The measurement chamber or column 54 has a base 160 generally including various apertures and conduits through which liquid enters and exits as described hereinbelow, as well as a top 162 including apertures for various probes, likewise described hereinbelow. A large aperture (not shown) is cut into the top of the fluid-handling subsystem 56 housing (not shown) for receiving the measurement chamber 54, and the base 160 is conveniently attached to the top of the fluid-handling subsystem 56 housing by bolted ferrules (not shown).

Internal to the measurement chamber 54 is a float switch 164, the function of which is to act as a fill control device for both sample and flush water charges. A Model M8000 float switch made by Madison Manufacturing Company may be employed.

The measurement chamber 54 has two drains, a main drain 166 centrally located in the base 160 and controlled by a drain solenoid valve 168 connected to a drain line 170, as well as a precautionary overflow tube or drain 172, connected directly to the drain line 170. The sample level controlled by the float switch 164 is purposely kept slightly below the overflow drain 172 to allow headspace for ISA fluids needed for conductivity adjustment. However, flushwater is allowed to purposely reach slightly above the overflow drain 172 so as to thoroughly clean out the entire chamber 54 inside surface. This is accomplished by programming that adds a few additional seconds of flushwater inlet “ON” time beyond the normal float-controlled “OFF” time.

Liquid primarily enters the measurement chamber 54 through four ports or openings within the base 160. More particularly, these are a sample input port 174 supplied by a sample pump 176 which draws from the sample flow-through reservoir 66, an ISA inlet port 178 supplied by an ISA pump 180 which draws from the ISA liquid reservoir 68, and an adjust water inlet port 182 supplied by an adjust water pump 184, which draws from the adjust water reservoir 70. Gorman-Rupp Model 16000-124 Bellows pumps may be employed. In addition, there is a rinse water or flush water inlet 186 which is supplied through a rinse solenoid valve 188 directly from the water line via the FIG. 1 connecting line 90.

In order to maintain a flow of liquid past various sensing electrodes described hereinbelow, required to maintain the proper operation thereof, a recirculation loop 190 is provided, including a recirculation pump 192, a recirculation liquid inlet 194, and a recirculation return 196. A Gormann-Rupp Model EX315-432 centrifugal pump is used in the disclosed embodiment, but any pump of similar size and performance specifications may be employed.

Projecting into the measurement chamber 54 through respective apertures in the top 162 are four probes, each described in greater detail hereinbelow with reference to FIGS. 6 and 7. These are a conductivity probe 200 which passes through an aperture 202, an ion-specific electrode 204 which passes through an aperture 206, a reference electrode 208 which passes through an aperture 210, and a re-standardizing reference electrode 212 which passes through an aperture 214. As shown in greater detail in FIG. 6, the conductivity probe 200 includes a pair of spaced conductivity-sensing electrodes 216 and 218 to which AC voltage is applied, as well as a temperature sensing element 220, preferably a resistance temperature detector (RTD).

Finally, an electric heater 222 is mounted to the base 160, and projects into the measurement chamber for heating a sample volume of liquid contained therein first to a predetermined conductivity measurement temperature, and sub subsequently to a predetermined concentration measurement temperature.

In operation of the apparatus 50 as thus far described, under control of the microcontroller 64, which activates the sample pump 176, a precisely metered volume of liquid from the sample flow-through reservoir enters the measurement chamber 54 until signaled by the float switch 164. This delivers a sample volume of liquid to the measurement chamber 54. The conductivity of the sample volume within the measurement chamber 54 is adjusted to a predetermined conductivity at a predetermined conductivity measurement temperature, employing the conductivity probe 200 in separate temperature control and conductivity control feedback loops. Although it is possible for the predetermined conductivity measurement temperature to simply be the ambient temperature within which the apparatus 50 is being operated, preferably the heater 222 is employed to heat the sample volume up to a temperature of, for example, 23.5° C., as the predetermined conductivity measurement temperature. The conductivity of the sample volume is adjusted to a conductivity which is consistent with the conductivity of the liquid stream being sampled. As an example, for color bleach fixers, the predetermined conductivity may be 55.0 milli-Siemens per centimeter. More particularly, the conductivity is adjusted by operation of the conductivity controller 152, operating in combination with the microcontroller 64, which together, as the automatic controller, operate the ISA pump 180 and the adjust water pump 184, to deliver quantities of ISA liquid and possibly adjust water from the ISA liquid reservoir 68 and the adjust water reservoir 70, as required.

When conductivity adjustment is completed, the sensing electrodes 216 and 218 of the conductivity probe 200 are electrically disconnected and shorted together in order to prevent them from influencing the subsequent ion concentration measurement. To allow all stray voltages to dissipate from the AC voltage applied to the conductivity sensing electrodes 216 and 218, there is a 60-second delay before a routine begins which involves employing the ion-specific electrode 204 and the reference electrode 208 to measure concentration of the ions of the specific metal. At the beginning of the concentration measurement routine, the liquid within the measurement chamber 54 is heated a second time, for example to 24.25° C. The ion-specific electrode typically requires time to achieve voltage stability, and a five-minute sample dwell time is inserted to allow the voltage measurement to become stable. Just prior to making a voltage measurement to acquire data, the heater 216 is operated for a third time, to bring the temperature of the liquid within the measurement chamber 54 to the predetermined conductivity measurement temperature, in this embodiment, 25° C. The temperature sensor 220 within the conductivity probe 200 is employed in each case to sense the temperature to determine when the heater 222 should be de-activated.

During this entire time, the recirculation pump 192 operates to maintain a flow of liquid past the ion specific and reference electrodes 204 and 208, respectively. Recirculation provided by the pump 192 is important to more quickly blend the relatively low volume of conductivity adjustment liquids into the relatively higher volume of sampled liquid already present within the measurement chamber 54, enabling the measurement process to proceed more quickly and efficiently.

In addition, as described hereinbelow with reference to FIG. 7, the ion-specific electrode 204 is periodically re-standardized.

Under control of the microcontroller 64 aided by the conductivity controller 152, which together serve as the automatic controller 154, these steps briefly described hereinabove are repeated at period intervals.

It is good practice to flush out the complete fluid system the measurement chamber 54 and recirculation loop 190 several times, especially if small (parts per billion) (ppb) levels are to be detected. In the example of monitoring silver ion concentration, it takes very little contamination by a prior ppm Ag liquid to contaminate a system in ppb Ag. Experience has shown that, regardless of starting residual silver left in the system after dumping, seven to ten water flushes clears out the Ag+ion down to undetectable levels. Multiple flushing also offers the side of minimizing the build-up of solid waste precipitates which could eventually foul the sensors, leading to erratic sensor readings.

For this reason, under microcontroller 64 program control, the measurement chamber 54 and recirculation loop 190 preferably are flushed out totally at least seven to ten times between cycles, seven times where “parts per million” (ppm) Ag limits are in effect, but at least ten times when the silver limit is expressed in “parts per billion” (ppb). The Gorrman-Rupp Model EX315-432 high-volume centrifugal pump employed as the recirculation pump 192 aids in rapidly flushing the system, to minimize downtime while the system is being flushed.

To protect the somewhat delicate sensor heads from drying out (thus rendering them inoperable), the operational cycle is set up to start with the measurement chamber 54 full of water. Thus, the last flushwater remains within the measurement chamber 54 until the “time to take a reading” programming sub-routine is reactivated.

With reference now to FIG. 5, the microcontroller 64 in the illustrated embodiment is a TFX-11v2, manufactured by Onset Computer Corporation, Bourne, Mass. The TFX-11v2 includes a number of digital I/O ports, as well as a number of 12-bit A/D converter input ports, program memory and data storage memory. Programming within the microcontroller to effect various functions described herein is represented by the flowcharts of FIGS. 9-23, and those FIGS. 9-23 should be referred to for additional details. A number of subsystems are connected to and controlled by the microcontroller 64. For convenience of illustration, the various subsystems are somewhat arbitrarily represented in FIG. 5 as boxes 240, 242 and 244, which represent the separate FIGS. 6, 7 and 8, respectively. In each of FIGS. 6, 7 and 8, a portion of the input and output lines of the microcontroller 64 are illustrated. It will be appreciated that the combination electrical schematic and functional block diagrams of FIGS. 6, 7 and 8 for purposes of illustration omit a number of conventional interfacing components. For example, various digital outputs of the microcontroller 64 are buffered through field-effect transistors (FETs) (not shown).

The block diagram of FIG. 5 also includes a local modem through which the microcontroller 64 transmits data. The local modem is connected via a telephone line represented at 248 to a remote modem 250 connected to a remote computer device 252, which serves as a data-recording file server, and may be off site.

With reference to FIG. 6, the conductivity controller 152 more particularly comprises a Eutech Model Con200 conductivity controller. However, any similar single-point two-relay conductivity controller with equal or better performance specifications may be employed. The temperature compensation function of the Eutech Model Con200 conductivity controller 152 is not employed. Instead, the temperature sensor 220 (which is part of the conductivity probe) and heater 222 are employed separately, under control of the microcontroller 64, to heat the sample volume to the predetermined concentration measurement temperature.

A suitable conductivity probe 200 is a Phoenix Electrode Model 2771314-31-003T Conductivity Probe. However, any conductivity probe with a Cell Constant of 1, a built-in 32K RTD temperature sensor, and having equal or better performance specifications may be employed.

The conductivity controller 152 more particularly includes a pair of probe terminals 260 and 262 which are connected to the conductivity-sensing electrodes 216 and 218 within the conductivity probe 200. The Eutech Model Con200 conductivity controller 152 applies a low voltage AC to the conductivity sensing electrodes 216 and 218 via the probe terminals 260 and 262 and measures the resultant current flow in order to determine conductivity. The conductivity controller 152 has a pair of analog output terminals 264 and 266 which drive a current representative of measured conductivity, in addition to providing a display output on the FIG. 2 front panel. This conductivity-indicating current output is converted to a voltage drop across a resistor 268, and sensed via an analog input 270 of the microcontroller 64 for data logging purposes.

The Eutech Model Con200 conductivity controller has a pair of relay outputs 272 and 274 having normally-open contact of respective relays 276 and 278 internal to the conductivity controller 152. In the illustrated embodiment, a common pole of the relays 276 and 278 is connected at 280 to a +12 volt supply.

During operation, the conductivity controller 152 activates the relay 276 when more ISA fluid is required in order to increase the conductivity of a sample, and the relay 278 when water is required to decrease the conductivity of a sample. Water may be required to correct a slight “overshoot” in the injection of ISA fluid.

Operation of the conductivity controller 152 is enabled by the microcontroller 64 so that operation of the conductivity controller 152 occurs only at appropriate times. Accordingly, the microcontroller 64 has digital outputs 282 and 284 connected (through appropriate buffers, not shown) to activate respective relays 286 and 288. The contacts of the relay 286 are arranged so as to connect the conductivity sensing electrodes 216 and 218 to the probe terminals 260 and 262 of the conductivity controller 152 only when the relay 286 is activated, and to otherwise entirely disconnect the conductivity sensing electrodes 216 and 218 from the rest of the circuitry and to short the two electrodes 216 and 218 together. Similarly, contacts of the relay 288 are arranged so as to operationally connect the relay outputs 272 and 274 of the conductivity controller 152 only when the relay 288 is activated.

More particularly, the relay output 272 is connected, through contacts of the relay 288, to drive a DPST relay 290. One pole of the relay 290 is connected to drive the “ISA pump” LED 132, and the other pole is connected to drive a solid state relay 292, which in turn energizes the ISA pump 180 which operates at 117 volts.

Likewise, the relay output 274 is connected through contacts of the relay 288, to drive a DPST relay 294. One pole of the relay 294 is connected to drive the “H₂O pump” LED 134, and the other pole is connected to drive a solid state relay 296, which in turn energizes the adjust water pump 184.

In addition, so that the microcontroller 64 can monitor and record the times when the ISA pump 180 and the adjust water pump 184 are operated, a pair of optical couplers 298 and 300 are also connected to the output of the solid state relays 292 and 296, respectively, and drive digital inputs 302 and 304 of the microcontroller 64.

FIG. 6 also represents circuitry for interfacing for the temperature sensor 220 to an A/D converter analog input 306 of the microcontroller 64 for temperature measurement purposes. More particularly, temperature measurement circuitry 308 includes a resistor network 310 and an appropriate amplifier circuit 312 having an input 314 and an output 316 connected to the microcontroller 64 analog input 306. Operation of the temperature measurement circuitry 308 is enabled by operation of a DPDT relay 318 controlled by a digital output 320 of the microcontroller 64. The contacts of the DPDT relay 318 are arranged, when not energized, to ground the input 314 of the amplifier 312, and to entirely disconnect the resistor network 310 and the temperature sensor 220 from the circuit. When activated, the contacts of the relay 318 are arranged so as to apply +5 volts to the resistor network 310, and connect an output of the resistor network 310 to the input 314 of the amplifier 312.

FIG. 7 represents circuitry for connecting the ion-specific electrode 204 and the reference electrode 208 to a 12-bit A/D converter analog input 350 of the microcontroller 64 for conductivity measurement purposes. A low-noise amplifier and signal conditioning circuit 352 has a pair of differential inputs 354 and 356 which receive and amplify the voltage across the electrodes 204 and 208, which voltage represents the concentration of specific metal ions within the sample volume. The amplifier circuit 352 has an output 358 connected to the microcontroller 64 analog input 350. In addition, FIG. 7 shows circuitry for re-standardizing the ion-specific electrode by applying a voltage first of one polarity then of opposite polarity to the ion-specific electrode 204 with reference to the re-standardizing reference electrode 208.

Three relays, a 4PDT relay 362, a 4PDT relay 364 and a DPDT relay 366, activated by respective digital outputs 368, 370 and 372 of the microcontroller 64 are employed to set up different operational states. When all three relays 362, 364 and 366 are deactivated, the ion-specific and reference electrodes 204 and 208 are shorted to each other and electrically disconnected from the circuit through contacts of the relays 364 and 362. The re-standardizing reference electrode 212 is entirely electrically disconnected from the rest of the circuitry. Inputs 354 and 356 of the amplifier 352 are connected to circuit ground via contacts of the relay 362.

To measure the voltage between the ion-specific electrode 204 and the reference electrode 208, which voltage is representative of the concentration of specific metal ions within the sample volume, the microcontroller 64 activates the 4PDT relay 362, which electrically connects the electrodes 204 and 208 to the inputs 364 and 366 of the amplifier 368. The resultant voltage is then recorded by the microcontroller 64 as a Test Voltage.

More particularly, a software routine executes which, in the case of Test minus Control (T-C) data, subtracts the Test Voltage from its paired Control voltage, and the resultant Test minus Control (T-C) voltage differential is then entered into a ppm vs diferential voltage calibration equation, which converts this differential voltage value to ppm silver. However, when using the Known Addition voltage measurement protocol, the actual Test measured voltage is entered into a ppm silver vs silver sensor voltage calibration equation, which directly converts the Test measured voltage into ppm silver.

For reference purposes, these data are stored within the server program (FIG. 6) permanently, but yet can be accessed anytime. A phoneline datalink and programming furnished to customers allows customers to access the data on their own computers in many formats, but typically displayed as a graph of ppm Silver versus Time (in days, weeks, or months).

To re-standardize the ion-specific electrode 204, the microcontroller 64 first activates the DPDT relay 366 which, in combination with the contacts of the relay 364, connects the re-standardizing reference electrode 212 to circuit ground and the ion-specific electrode 204 to the output of a +3 volt power supply 376, for about three seconds. A contact of the relay 364 also drives the “Probe Clean” LED 128. Then, to apply a voltage of opposite polarity to the ion-specific electrode 204 with reference to the re-standardizing reference electrode 212, the relay 366 is activated, in addition to the relay 364, thereby connecting the ion-specific electrode 204 to circuit ground, and the re-standardizing reference electrode 212 to the output of the +3 volt power supply 376.

Due to a limited number of output lines of the microcontroller 64, the digital output 372 is also employed to activate a relay 380 which has contacts arranged to drive a power relay 382 which in turn drives the heater 222, in addition to contacts arranged to activate the “heater on” LED 130.

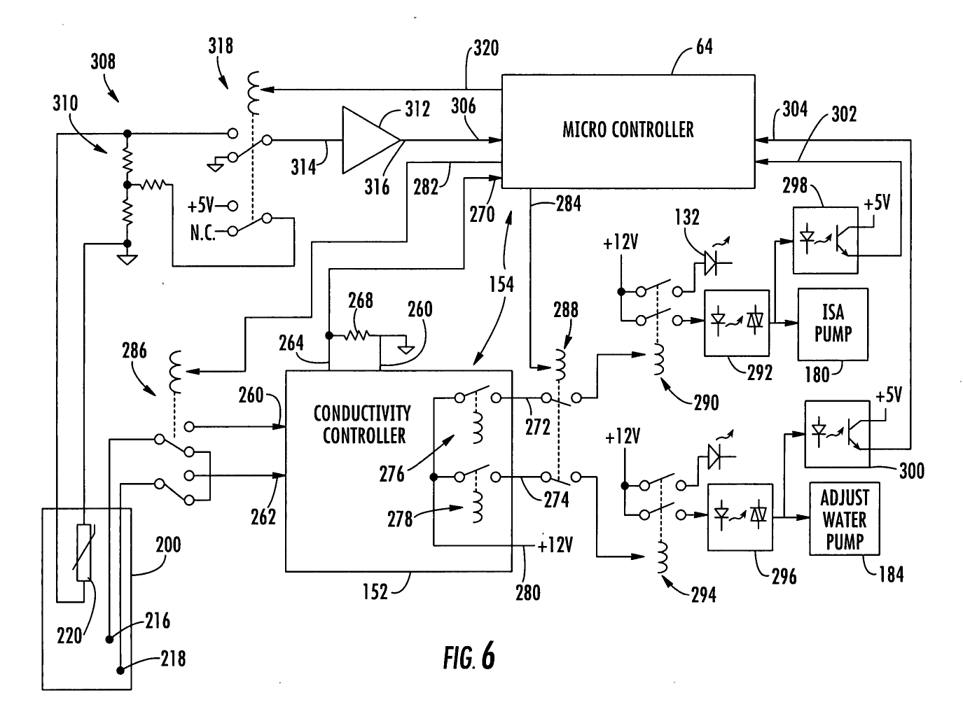

FIG. 8 represents connections of four additional digital control outputs of the microcontroller 64, for activating each of the sample pump 176, the recirculation pump 192, the rinse solenoid valve 188 and the drain solenoid valve 168. Digital outputs 390, 392, 294 and 296 of the microcontroller 64 activate respective DPST relays 400, 402, 404 and 406. One pole of the relay 400 is connected to drive the “Sample pump” LED 138, and the other pole is connected to drive a solid state relay 410, which in turn energizes the sample pump 176. One pole of the relay 402 is connected to drive the “Recirculation pump” LED 136, and the other pole is connected to drive a power relay 412, which in turn energizes the recirculation pump 192. One pole of the relay 404 is connected to drive the “Rinse Solenoid” LED 142, and the other pole is connected to drive a power relay 414, which in turn energizes the rinse solenoid valve. Finally, one pole of the relay 406 is connected to drive the “Drain Solenoid” LED 140, and the other pole is connected to drive a power relay 416, which in turn energizes the drain solenoid valve 168.

The microcontroller 64 additionally has “marginal” and “fail” digital outputs 420 and 422 which can optionally be employed to shut down an associated process in the event measured concentration exceeds predetermined limits. These outputs are connected to a user-operable switch 424 which selects one or the other to drive an output relay that can be used to shut down the associated equipment, such as a photo-processor.

The embodiment described herein is particularly for measuring the concentration of silver ions, and the ion specific electrode 204 accordingly is a potentiometric half-cell responsive to the concentration of silver ions. A Model AGS1501-003B Silver/Sulfide ISE sensor available from phoenix Electrode Company may be employed. As the reference electrode 208, another ion specific electrode or potentiometric half-cell which is specific to ions which are not expected to be present in the stream from which the sample volume is taken advantageously may be employed. In the disclosed embodiment, a Model F001501-003B Lanthanum Flouride ISE sensor, also available from phoenix Electrode Company is employed as the reference electrode 208.

As the re-standardizing reference electrode 212, a stainless steel rod may be employed.

Standard statistical techniques are used to remove all wild points from the data collected for all Test samples. An “average value” is then calculated for each of these samples. These average values are then corrected, if necessary, for minor aberrations in temperature and/or conductivity, and converted to ppm silver (by a previously generated silver volts-to-ppm silver equation. If that ppm Silver value falls within the pre-set limit (say 5.0 ppm), then the system is considered performing within spec, the number is sent by modem to an off-site file server and stored in a file against a date & time stamp.

However, if the value obtained is outside the limit, then in addition to being stored in the mainframe server file, that particular value also enters a special file that tracks “out-of-limit” performance. In order for an “Out-of-Limit” notification to be given, this sub-routine must recognize at least three “out-of-limit” values in sequence; otherwise the sub-routine resets itself. This “Three Strikes and your Out” approach ensures that the customer never receives a false positive indication.

Actually, contained within this sub-routine is yet another sub-routine that requires three valid “out-of-limit” indications before that “Strike” is validated. Therefore, with this approach, there must be at least nine valid “out-of-limit” responses before an “Out-of-Limit” notification is given. This technique is not the same as just nine “out-of-limit” values in sequence, because any one “within-limit” value merely resets that particular strike, but all strikes validated ahead of this strike remain in place.) This technique minimizes false alarms.

Also, four or more sequential “within-limit” values reset all Strikes, the assumption being that whatever the problem was has been corrected. Of course, even if this resetting were to take place, the individual “out-of-limit” data is not lost to the customer, as each of these points would show up on any historical PPM Silver vs Time plot, should one be generated that covers that particular time and date.

An especially good technique to use where very small ppm silver levels are to be monitored (say 1 ppm or less) is the use of Standard Addition (also called Known Addition), where a small amount of a known silver solution is added to the sample, followed by a second reading, after the voltage has re-stabilized in the mixture.

The volume and concentration of the standard must be carefully chosen to meet the following criteria:
1) The concentration and volume of the additive must be sufficient to cause a significant and measurable change of the measured voltage of the sample solution, but the volume must also be small enough so that it does not cause a significant change in the sample Ionic Strength.
2) The volume of the additive must be large enough so that volumetric errors are not significant.

Both of these criteria are easily met with the disclosed system, with appropriate programming, enabled by toggling a “Known Addition Activated? Yes/No” flag upon system startup.

An advantage of using this technique is that both test and control measurements are made while the electrodes are continually immersed in fluids sharing the same (or very nearly the same) temperature and Ionic Strength.

While specific embodiments of the invention have been illustrated and described herein, it is realized that numerous modifications and changes will occur to those skilled in the art. It is therefore to be understood that the appended claims are intended to cover all such modifications and changes as fall within the true spirit and scope of the invention.

1. A method for monitoring concentration of ions of a specific metal in solution in a liquid process or liquid waste stream, comprising employing an automatic controller to:
deliver a sample volume of liquid from the stream to a measurement chamber;
adjust the conductivity of the sample volume within the measurement chamber to a predetermined conductivity at a predetermined conductivity measurement temperature;
employ an ion specific electrode and a reference electrode to make a voltage measurement representative of the concentration of specific metal ions within the sample volume at the predetermined concentration measurement temperature;
record the voltage measurement; and
flush the measurement chamber.

2. The method of claim 1, which comprises employing the automatic controller to periodically at predetermined intervals cycle through steps of delivering, adjusting, employing and flushing.

3. The method of claim 1, which further comprises employing the automatic controller to transmit the voltage measurement to a remote location for recording.

4. The method of claim 3, which comprises employing the automatic controller to periodically at predetermined intervals cycle through steps of delivering, adjusting, employing, transmitting and flushing.

5. The method of claim 1, which further comprises employing the automatic controller to heat the delivered sample volume to the predetermined conductivity measurement temperature at least prior to adjusting the conductivity of the sample volume within the measurement chamber to a predetermined conductivity, and to heat the delivered sample volume to the predetermined concentration measurement temperature prior to employing the ion specific electrode and the reference electrode to make a voltage measurement representative of the concentration of specific metal ions.

6. The method of claim 1, wherein employing the automatic controller to adjust the conductivity of the sample volume comprises employing a conductivity sensor within a feedback loop to control the addition of ionic strength adjuster liquids to the measurement chamber.

7. The method of claim 6, wherein employing the automatic controller to employ the ion specific electrode and the reference electrode to make a voltage measurement representative of the concentration of the specific metal ions further comprises electrically disconnecting the conductivity sensor.

8. The method of claim 1, which further comprises employing the automatic controller to re-standardize the ion specific electrode by delivering water to the measurement chamber, and then for predetermined durations, to apply voltages first of one polarity and then of opposite polarity to the ion specific electrode with reference to a re-standardizing reference electrode.

9. The method of claim 8, wherein re-standardizing the ion specific electrode comprises employing a stainless steel electrode as the re-standardizing reference electrode.

10. The method of claim 8, wherein employing the automatic controller to employ the ion specific electrode and the reference electrode to make a voltage measurement representative of the concentration of the specific metal ions further comprises electrically disconnecting the re-standardizing reference electrode.

11. The method of claim 1, wherein employing the automatic controller to employ an ion specific electrode to make a voltage measurement representative of the concentration of the specific metal ions comprises employing a pair of potentiometric half-cells as the ion specific electrode and as the reference electrode.

12. The method of claim 11, wherein employing the automatic controller to employ an ion specific electrode to make a voltage measurement representative of the concentration of the specific metal ions comprises employing as the reference electrode another ion specific electrode which is specific to ions which are not expected to be present in the stream from which the sample volume is taken.

13. The method of claim 1, which is for monitoring the concentration of silver ions in solution in the liquid process or liquid waste stream, wherein employing the automatic controller to adjust the conductivity of the sample volume comprises employing a conductivity sensor within a feedback loop to control the addition of an ammonium thiosulfate solution as an ionic strength adjuster to the measurement chamber.

14. The method of claim 1, which is for monitoring the concentration of silver ions in solution in the liquid process or liquid waste stream, wherein employing the automatic controller to employ an ion specific electrode and a reference electrode comprises employing a silver ion specific electrode as the ion specific electrode.

15. The method of claim 14, which comprises employing a silver sulfide electrode as the silver ion specific electrode.

16. The method of claim 1, which is for monitoring the concentration of silver ions in solution in the liquid process or liquid waste stream, wherein employing the automatic controller to employ an ion specific electrode and a reference electrode comprises employing a lanthanum fluoride electrode as the reference electrode.

17. A method for re-standardizing an ion specific electrode, comprising, for predetermined durations, applying voltages first of one polarity and then of opposite polarity to the ion specific electrode with reference to a re-standardizing reference electrode.

18. Apparatus for monitoring concentration of ions of a metal in solution in a liquid process or liquid waste stream, comprising:
a measurement chamber;
a conductivity sensor within said measurement chamber;
an ion specific electrode and a reference electrode within said measurement chamber;
a controlled sample injection pump for delivering liquid from the stream to said measurement chamber;
a controlled ISA injection pump for delivering an ionic strength adjuster liquid to said measurement chamber;
a controlled water pump for delivering water to said measurement chamber;
a controller electrically connected to receive signals from said conductivity sensor and from said ion specific and reference electrodes and connected to control operation of said sample injection pump, said ISA injection pump and said water pump, said controller operable to effect a measurement cycle by
directing said sample injection pump to deliver a sample volume of liquid from the stream to said measurement chamber,
adjusting the conductivity of the sample volume within said measurement chamber to a predetermined conductivity at a predetermined conductivity measurement temperature by employing said conductivity sensor and said ISA injection pump in a feedback loop,
making a voltage measurement representative of the concentration of specific metal ions in the sample volume at a predetermined concentration measurement temperature by employing said ion specific electrode and said reference electrode, and recording the voltage measurement, and
flushing said measurement chamber.

19. The apparatus of claim 18, wherein said controller is operable to effect the measurement cycle periodically at predetermined intervals.

20. The apparatus of claim 18, which further comprises a communications device for transmitting the voltage measurement to a remote location for recording.

21. The apparatus of claim 18, which further comprises a heater for liquid within said measurement chamber.

22. The apparatus of claim 21, wherein said heater is operable to heat liquid within said measurement chamber to the predetermined conductivity measurement temperature at least prior to adjusting the conductivity of the sample, and operable to heat liquid within said measurement chamber to the predetermined concentration measurement temperature prior to making a voltage measurement representative of the concentration of specific metal ions in the sample volume.

23. The apparatus of claim 18, wherein said controller is further operable to adjust the conductivity of the sample volume within said measurement chamber to a predetermined conductivity by employing said conductivity sensor, said ISA injection pump and said water pump within a feedback loop.

24. The apparatus of claim 18, wherein said an ion specific electrode and said reference electrode comprise a pair of potentiometric half-cells.

25. The apparatus of claim 24, wherein said reference electrode comprises another ion specific electrode which is specific to ions which are not expected to be present in the stream from which the sample volume is taken.

26. The apparatus of claim 18, wherein the ionic strength adjuster is an ammonium thiosulfate solution.

27. The apparatus of claim 18, which is for monitoring the concentration of silver ions in solution in the liquid process or liquid waste stream, wherein said ion specific electrode comprises a silver ion specific electrode.

28. The apparatus of claim 27, wherein said ion specific electrode comprises a silver sulfide electrode.

29. The apparatus of claim 18, which is for monitoring the concentration of silver ions in solution in the liquid process or liquid waste stream, wherein said reference electrode comprises employing a lanthanum fluoride electrode.

30. The apparatus of claim 18, which further comprises a recirculation pump connected to said measurement chamber for maintaining a flow of liquid past said electrodes.

31. The apparatus of claim 18, which further comprises a re-standardizing reference electrode within said measurement chamber, and wherein said controller is further operable to periodically re-standardize said ion specific electrode by directing said water pump to deliver water to said measurement chamber, and then for predetermined durations, cause applying voltages first of one polarity and then of opposite polarity to be applied to said ion specific electrode with reference to said re-standardizing reference electrode.

32. The apparatus of claim 18, which further comprises a flow-through sample reservoir having an input for receiving the liquid stream and an overflow outlet, said sample injection pump being connected for drawing liquid from said flow-through sample reservoir.

33. The apparatus of claim 32, which comprises a self-contained unit additionally including an ionic strength adjuster reservoir from which said ISA injection pump draws ionic strength adjuster liquid, and a water reservoir from which said water pump draws water.

34. A system for monitoring concentration of ions of a metal in solution in a liquid process or liquid waste stream, comprising:
a local device including
a measurement chamber,
a conductivity sensor within said measurement chamber;
an ion specific electrode and a reference electrode within said measurement chamber,
a controlled sample injection pump for delivering liquid from the stream to said measurement chamber;
a controlled ISA injection pump for delivering an ionic strength adjuster liquid to said measurement chamber;
a controlled water pump for delivering water to said measurement chamber,
a controller electrically connected to receive signals from said conductivity sensor and from said ion specific and reference electrodes and connected to control operation of said sample injection pump, said ISA injection pump and said water pump, said controller operable to effect a measurement cycle by
directing said sample injection pump to deliver a sample volume of liquid from the stream to said measurement chamber,
adjusting the conductivity of the sample volume within said measurement chamber to a predetermined conductivity at a predetermined conductivity measurement temperature by employing said conductivity sensor and said ISA injection pump in a feedback loop,
making a voltage measurement representative of the concentration of specific metal ions in the sample volume at a predetermined concentration measurement temperature by employing said ion specific electrode and said reference electrode, and recording the voltage measurement, and
flushing said measurement chamber, and
a local communications device for transmitting the voltage measurement to a remote location for recording; and
a remote computer device at the remote location for receiving and recording the voltage measurement.
